(19) United States
(12) Patent Application Publication
(10) Pub. No.: US 2005/0059068 A1
(43) Pub. Date: Mar. 17, 2005

(54) Compositions and methods using dendrimer-treated microassays

(75) Inventors: Haoqiang Huang, San Diego, CA (US); Jeffrey Carl Braman, Carlsbad, CA (US)

Correspondence Address: PALMER & DODGE, LLP;KATHLEEN M. WILLIAMS / STR, 111 HUNTINGTON AVENUE, BOSTON, MA, 02199, US

(73) Assignee: Stratagene California

(21) Appl. No.: 10/938,807

(22) Filed: Sep. 10, 2004

Related U.S. Application Data
(63) Continuation of application No. 09/863,748, filed on May 23, 2001, now abandoned.

(51) Int. Cl. C12Q 1/68; G01N 33/53; C12M 1/34

(52) U.S. Cl. 435/6; 435/7.1; 435/287.2

(57) ABSTRACT

The present invention provides a chemically reactive surface able to covalently react with substances containing a hydroxyl group and/or amine group, comprising a solid surface having an activated dendrimer polyamine covalently bonded to said surface through a silane containing reagent, wherein the dendrimer polyamine can covalently bind the substance comprising a hydroxyl group and/or amino group. The present invention further provides a method for producing chemically reactive surfaces for binding moieties comprising a hydroxyl group and/or amine group, as well as kits comprising the chemically reactive surface of the invention.

### BACKGROUND OF THE INVENTION

Microarray technology represents an exciting advancement in the field of biology. It offers massive parallel data accumulation and analysis while significantly reducing time and experimental materials. Since its emergence in 1993, there has been explosive growth in microarray development and applications. More than 40 companies are now involved in this field with annual sales of between 300 to 400 million dollars. The estimated market is expected to reach close to one billion dollars per year within the next five years (Gabriel (1999) Biomed. Prod. 10: 26). As the technology becomes more widely accessible, a large number of researchers will be able to shift their focus from a linear study of individual biochemical events to matrix analysis of complex systems and pathways (Cortese (2000) The Scientist, 25).

Several materials have been used for fabricating microarrays including glass microscope slides, nylon membranes, plastic slides and polymer matrices such as polyacrylamide. (Schea (Ed.) Microarray Biochip Technology, 2000, Eaton Publishing, Mass.; Beier and Hoheisel (1999) Nucleic Acids Research, 27:1970) These materials permit immobilization of large amounts of probe molecules and provide a favorable environment for molecular interactions to occur (Dubiley et al., (1997) Nucleic Acids Res. 25: 2259; Yershov et al., (1996) Proc. Natl. Acad. Sci. USA 93: 4319). However, nylon is somewhat fragile and can be damaged by devices making contact with them during microarray printing, hybridization and scanning. Furthermore, capillary forces, inherent in nylon, results in wicking of liquid from contact dispensers, resulting in undesirable spreading of probe molecules, and poorly controlled dispensing volumes (Schena (Ed.) Microarray Biochip Technology 2000, Eaton Publishing, Mass.). Another disadvantage of nylon and plastic is high autofluorescence background. In addition, plastic materials like polypropylene cannot be used for in situ oligonucleotide synthesis because the ploymer swells in organic solvents This material is also not suitable for fluorescent confocal scanning (Beier and Hoheisel (1999) Nucleic Acids Res. 27: 1970).

Clear glass microscope slides are used for microarrays because they are compatible with existing microscopy tools, micro-spotting and ink-jet printing techniques, and commercial fluorescent scanning. Additional advantages of this convenient platform include low intrinsic fluorescence, ease of surface modification and simplicity of target hybridization and washing. Thin film hybridization on glass microscope slides allows low hybridization volumes (5-20 μl), high target concentration and rapid hybridization kinetics.

DNA probes are immobilized on glass surfaces through two different methods: “in situ” synthesis and “pre-synthesis, then spotting”. Affymetrix (Santa Clara, Calif.) uses photolithograph to synthesize up to 24 bases of single stranded DNA on the slide surface (U.S. Pat. Nos. 5,445,934; 5,744,305). The second method uses a variety of printing approaches developed by Ronald Davis and Patrick Brown at Stanford University (DeRisi et al., (1996) Nat. Genet. 14: 457; DeRisi et al., (1997) Science 278: 680; Heller et al.; (1997) Proc. Natl. Acad. Sci. USA 94: 2150) for binding of pre-synthesized oligonucleotides, cDNA and polymerase chain reaction (PCR) products.

Non-covalent attachment is commonly used for spotting cDNA and PCR product arrays. The glass slides used for this purpose may be treated with silane that creates a layer of free amine groups on the surface. The polyanionic backbone of DNA then interacts with the positively charged groups on the aminosilane surface. The attachment of probe DNA to this coating is relatively poor to weak signal and irreproducible results due to disassociation of probe DNA during hybridization and washing.

Another non-covalent attachment method employs coating slides with poly-L-lysine coating. The negatively charged probe DNA interacts with the positively charged poly-L-lysine surface (Schena (Ed.) supra). It may be possible to observe stronger signal on poly-L-lysine slides compared to aminosilane modified slides because of the higher amine density on polylysine resulting in higher DNA probe loading. However, unacceptable “streaking” between spots (FIG. 3A) and easily damaged surface limits this slide coating material.

Several methods of glass surface modification have been developed for creating activated functional groups allowing covalent binding of probe DNA. An aldehyde reactive surface is used to attach 5′-amine-terminated DNA via a Schiff's base reaction. However, the Schiff base is easily decomposed. Mercapto-silanes are another group of compounds used to modify the slide surface. 5′-amine bearing DNA is linked to sulfhydryl groups through a hetro-bifunctional cross linking reagent such as γ-maleimide-butyryloxy-succinimide (GMBS). Adessi et. al. (2000, Nucleic Acids Res. 28: e87) also reported coupling 5′-thiol modified DNA to amine groups on the glass surface in the presence of m-maleimido-benzoyl-N-succinimide ester (MBS). However, mercaptans are easily air oxidized so the mercaptosilanized slides or the 5′-thiol-modified DNA must be used as quickly as possible following modification to yield maximal probe immobilization (Schena, supra).

Yet another technique for binding probe DNA to glass involves attaching 5′-amine-terminated probe DNA through end-point attachment to polyacrylamide (Livshits and Mirjabekov 1996, Biophysical Journal 71:2795; Mirjabekov et al., U.S. Pat. No. 5,981,734). The advantage of using this approach is that diffusion of nucleic acid targets to acrylamide bound probe is poor, resulting in non-uniform hybridization within the gel pad and slow hybridization kinetics (Spangler 2000, Am. J. Med. Genet 96: 604).

Dendrimer coated glass slides have been described by Beier and Hoheisel (1999 Nucleic Acids Research 27:1970). They describe a process of synthesizing dendrimers on a glass surface, using tetraethylenpentamine as the core of the dendrimer. In contrast, the present invention discusses using a core amine having the structure NH₂(CH₂CH₂NH)ₙCH₂CH₂NH₂, where n is at least 4. The present invention also teaches the modification of a surface using a prefabricated dendrimer polyamine having either secondary or tertiary amines at the branch points of the dendrimer and amide linkages between successive generations of the dendrimer. The use of prefabricated dendrimer polyamines of this nature are not described by Beier and Hoheisel. Beier and Hoheisel further describe that their modified slides may be reused more than twice, provided that the components bound to the slide (i.e., DNA, or polypeptide) are 5′-amino modified. Beier and Hoheisel specifically mention that dendrimer treated slides reacted with unmodified nucleic acid, could not be reused without loss of signal. In contrast, the present invention shows that unmodified amine or hydroxyl containing compounds may be bound to the slide and reused more than three times without loss of signal or creation of excess background.

The major limitations of all commercialized surface modifications and target DNA binding protocols are the requirement for target DNA modification with 5′-amino or 5′-thiol groups, use of toxic chemicals by the customer to attach their nucleic acid to the surface, and the instability of the activated coated surface. There is a strong need to develop a convenient and customer-friendly surface modification and probe DNA binding technique, allowing array users to covalently immobilize unmodified cDNA, PCR products or oligonucleotides.

### SUMMARY OF THE INVENTION

The present invention provides a chemically reactive surface reactive with a substance comprising a hydroxyl group or amine group comprising: a solid surface having an activated dendrimer polyamine covalently bonded to the surface through a silane containing moiety, the dendrimer polyamine comprising branch points and terminal residues, a branch point of the dendrimer comprising either a secondary or a tertiary amine, a terminal residue comprising a moiety selected from the group consisting of primary amine, hydroxyl, carboxyl, and thiol, and wherein in the presence of a substance comprising a hydroxyl group or amine group, the activated dendrimer polyamine covalently binds the substance through the hydroxyl group or amine group. Depending on the heterobifunctional crosslinker, other functional groups like —SH could be bound to the glass slide.

In one embodiment, the surface is glass.

In a preferred embodiment, the glass surface is a glass slide.

In a further embodiment, the surface is a synthetic polymer material selected from the group consisting polypropylene, nylon, poly-styrene, poly-carbonate or other plastic polymer slides, poly-styrene, poly-carbonate or other plastic polymer well plates, beads, membranes, and glass wool.

In one embodiment, the silane containing moiety has the formula XR′Si(OR″)₃: wherein R′ is alkyl containing 0-10 carbons, R″ is alkyl containing 1-10 carbons, X is selected from the group consisting of NH₂, SH, OH, CN, halogen, methacrylate, quaternary amine salt, carboxylic acid and carboxylic acid salt, phosphonate, succinic anhydride, 2-carbomethoxyaziridine, dihydroimidazole, thiocyanato, isocyanato, isopropeno, 2,3-epoxypropoxy, and epoxy-alkyl.

In one embodiment, the dendrimer polyamine comprises [6]ⁿ terminal primary amine groups, wherein n=1, 2, 3, or 4.

In a further embodiment, the dendrimer polyamine comprises [15]ⁿ terminal primary amine groups, wherein n=1, 2, 3, or 4.

In a further embodiment, the dendrimer polyamine comprises [31]ⁿ terminal primary amine groups, wherein n=1, 2, 3, or 4.

In a still further embodiment, the dendrimer polyamine comprises [63]ⁿ terminal primary amine groups, wherein n=1, 2, 3, or 4.

In one embodiment, the substance comprising a hydroxyl group or amine group is selected from the group consisting of DNA, RNA, polypeptides.

In a preferred embodiment, the substance comprising a hydroxyl or amine group is covalently bonded to the dendrimer polyamine.

In one embodiment, the surface is chemically stable at room temperature.

In a preferred embodiment, the surface is chemically stable at room temperature for at least three months.

In a still further embodiment, the dendrimer polyamine comprises amide linkages between any two successive dendrimer generations and comprises a secondary or tertiary amine at a branch point that does not contain the amide linkage, wherein a branch of the dendrimer polyamine comprises a carbon-carbon bond, a carbon-oxygen-carbon bond, or a carbon-nitrogen-carbon bond.

The present invention further provides a kit comprising a chemically reactive surface reactive with a substance comprising a hydroxyl group or amine group, comprising: a solid surface having an activated dendrimer polyamine covalently bonded to the surface through a silane containing moiety, the dendrimer polyamine comprising branch points and terminal residues, a branch point of the dendrimer comprising either a secondary or a tertiary amine, a terminal residue comprising moiety selected from the group consisting of primary amine, hydroxyl, carboxyl, and thiol, and wherein in the presence of a substance comprising a hydroxyl group or amine group, the activated dendrimer polyamine covalently binds the substance through the hydroxyl group or amine group; and packaging materials therefore.

The invention further provides a kit comprising a chemically reactive surface reactive with a substance comprising a hydroxyl group or amine group, comprising: a solid surface having an activated dendrimer polyamine covalently bonded to the surface through a silane containing moiety, the dendrimer polyamine comprising branch points and terminal residues, a branch point of the dendrimer comprising either a secondary or a tertiary amine, a terminal residue comprising a moiety selected from the group consisting of primary amine, hydroxyl, carboxyl, and thiol, and wherein the substance comprising a hydroxyl group or an amine group is covalently bonded to the dendrimer polyamine; and packaging materials therefore.

In one embodiment, the kit further comprises nucleic acid printing buffer.

In one embodiment, the kit further comprises polypeptide printing buffer.

In one embodiment, the kit further comprises a nucleic acid hybridization solution.

In one embodiment, the kit further comprises deactivating solution.

In one embodiment, the kit further comprises a nucleic acid stripping solution

In one embodiment, the glass slide is maintained in an anhydrous state until contacted with a nucleic acid, or polypeptide and/or a nucleic acid or polypeptide printing buffer.

The invention also provides a method of making a chemically reactive surface which is reactive with a substance comprising a hydroxyl group or amine group, comprising: (a) contacting the surface with a silane containing moiety comprising a reactive functionality, under conditions to produce a silanized surface; (b) contacting the silanized surface with a reagent containing a terminally unsaturated carbon which chemically reacts with the reactive functionality to produce a surface capable of reacting with an amine group containing compound; (c) reacting the surface with a first amino group containing compound having the formula NH₂(CH₂)ₘY[(CH₂)ₙY]ₓ(CH₂)ₘNH₂, wherein m,n equals 1-15, x equals 4-15, and Y is O, or NH; (d) contacting the first amino group containing compound with a reagent containing a terminally unsaturated carbon to produce a surface capable of reacting with an amine group containing compound; reacting the reagent containing a terminally unsaturated carbon with a second amino group containing compound to produce a first dendrimer generation; (e) sequentially repeating the above steps (b) and (e) on the silanized surface so as to generate polyamine dendrimer that is chemically bonded to the surface comprising [6]ⁿ terminal primary amine groups, wherein n=1, 2, 3, 4, 5, or 6; (f) reacting the surface with a reagent which activates the amine groups so as to render the surface reactive with a substance comprising a hydroxyl group or amine group; wherein after each of steps (a)-(g), the surface is dried.

In one embodiment, the reactive functionality is an amine group.

In one embodiment, the reagent containing a terminally unsaturated carbon is selected from the group consisting of acryloylchloride, 4-nitrophenyl-chloroformate, acryloyliodide, acryloylbromide, 4-nitrophenyl-bromoformate, 4-nitrophenyl-iodoformate, CH₂═CRC(O)Cl, CHR═CHC(O)Cl, RR′C═CHC(O)Cl, CH₂═CH(CH₂)ₙC(O)Cl, wherein R or R′ equals 1-15 carbons and n equals 0-10.

In one embodiment, the second amine group containing compound is selected from the group consisting of: tetraethylenpentamine, 1,4-bis-(3-aminopropoxy)butane, 4-aminomethyl-1,8-octadiamine, 4,7,10-trioxa-1,13-tridecandiaamine, N,N-dimethyl-1,6-hexadiamine, 2-(2-aminoethoxy)ethanol, jeffamine 130, 3-amino-1,2-propandiol, hesandiamine, cyclohexandiamine, pentaethylenehexaamine, polyethylenepolyamine, and NH₂(CH₂CH₂NH)ₙCH₂CH₂NH₂, wherein n=2, 4, 5-12.

In one embodiment, the reagent which activates the amine groups is selected from the group consisting of: phenylendiisothiocyanate, disuccinimidylcarbonate, phenylendiisocyanate, disuccinimidyloxalate, bis-2-succinimido-oxycarbonyloxyethyl sulfone (BSOCOES), sulfo-BSOCOES, bis-sulfosuccinimidyl-disuccinimidyl tartarate (DST), sulfo-DST, ethylene glycol bis-succinimidylsuccinate (EGS), and dimethylsuberimidate.

In a preferred embodiment, the reagent which activates the amine groups is phenylendiisothiocyanate.

The present invention also provides a method of making a chemically reactive surface which is reactive with a substance comprising a hydroxyl group or amino group, comprising: (a) contacting the surface with a silane containing moiety comprising a reactive functionality, under conditions to produce a silanized surface; (b) contacting the silanized surface with a reagent containing a terminally unsaturated carbon which chemically reacts with the reactive functionality to produce a surface capable of reacting with a dendrimer polyamine; (c) reacting the silanized surface with a dendrimer polyamine selected from the group consisting of polypropylenimine hexadecaamine, polypropylenimine tetraamine dendrimer, polypropylenimine octaamine dendrimer, polypropylenimine hexadecaamine dendrimer, polypropylenimine dotriacontaamine dendrimer, or polypropylenimine tetrahexacontaamine dendrimer, wherein the dendrimer polyamine comprises terminal primary amine groups; (d) sequentially repeating the above steps (b) and (c) on the silanized surface so as to generate polyamine dendrimer that is chemically bonded to the surface comprising [5]ⁿ, [15]ⁿ, [31]ⁿ, or [63]ⁿ terminal primary amine groups, wherein n=1, 2, 3, or 4; and (e) reacting the surface with a reagent which activates the terminal primary amine groups so as to render the surface reactive with the substance comprising a hydroxyl group or amine group; wherein after each of the contacting or reacting steps the surface is dried.

In one embodiment, the method further comprises the step of reacting the surface with the substance comprising a hydroxyl group or amine group.

As used herein, a “chemically reactive surface” refers to any surface, useful in the present invention, to which may be bound a polyamine dendrimer. A “chemically reactive surface” further refers to a surface comprising a dendrimer polyamine to which may be bound a substance comprising a hydroxyl group or an amine group, such as nucleic acid or protein using reaction conditions well known to those of skill in the art.

As used herein, a “surface” generally refers to a two-dimensional structure on a solid substrate which may have steps, ridges, kinks, terraces, and the like without ceasing to be a surface. “Surfaces” useful in the present invention include, but are not limited to glass, poly-styrene, poly-carbonate or other plastic polymer slides, poly-styrene, poly-carbonate or other plastic polymer well plates, beads, membranes, glass wool, and other solid support materials for combinatorial chemistry reactions.

As used herein, “dendrimer polyamine” refers to a macromolecular polymer with regular, dendritic branching with radial symmetry composed of an initiator core, interior layers (or generations) of repeating units, radially attached to the core and an exterior surface of terminal functional groups. The chemical structure of a dendrimer polyamine is well known to those of skill in the art (See: D. A. Tomalia and H. D. Durst (1993) in E. Weber (ed.) Topics in Current Chemistry, Vol. 165: Supramolecular Chemistry I-Directed Synthesis and Molecular Recognition, Springer-Verlag, Berlin, pp.193-313). The terminal functional groups of “dendrimer polyamines” useful in the present invention can be primary amine groups, hydroxyl groups, carboxyl groups or protected thiol groups. “Dendrimer polyamines” useful in the present invention further comprise one or more amide linkages between successive dendrimer generations and secondary or tertiary amines at the branch points that do not comprise amide linkages, wherein the branch point comprises carbon-carbon bonds, carbon-oxygen-carbon bonds, or carbon-nitrogen-carbon bonds. A “dendrimer polyamine” useful in the present invention may include, but may not be limited to polypropylenimine hexadecaamine, polypropylenimine tetraamine dendrimer, polypropylenimine octaamine dendrimer, polypropylenimine hexadecaamine dendrimer, polypropylenimine dotriacontaamine dendrimer, and polypropylenimine tetrahexacontaamine dendrimer.

As used herein, a “hydroxyl group” refers to a chemical group having the structure
X—OH
wherein X is any molecule, O is oxygen, and H is hydrogen.

As used herein, an “amine group” refers to a chemical group having the structure
X—NH₂
wherein X is any molecule, N is nitrogen, and H is hydrogen.

As used herein, “covalently bonded” refers to the interaction between two chemical moieties wherein electrons are shared by the atomic nuclei of the two moieties, thus bonding the two moieties together.

As used herein, a “silane containing moiety” refers to a gaseous or liquid compound of silicon and hydrogen, analogous to alkanes or saturated hydrocarbons, wherein the “silane containing moiety” has the ability to bond organic polymer systems to inorganic substrates. “Silane containing moieties” useful in the present invention have the general formula XR′Si(OR″)₃, where R′ is alkyl containing 0-10 carbon atoms, R″ is alkyl containing 1-10 carbon atoms, Si is silicon, and X is referred to as a “reactive functionality”, and includes, but is not limited to NH₂, SH, OH, CN, halogens, methacrylate, quaternary amine salts, carboxylic acids and salts, phosphonates, succinic anhydride, 2-carbomethoxyaziridine, dihydroimidazole, thiocyanato, isocyanato, isopropeno, 2,3-epoxypropoxy, epoxy-alkyl, and the like.

As used herein “unmodified” as it refers to nucleic acid or peptides refers to the nucleic acid or peptide being in its natural state, without any alterations made to structure, or atomic components following isolation and purification, or synthesis.

As used herein “chemically stable” refers to a property of the “chemically reactive surfaces” of the present invention wherein the chemically reactive surface retains its chemical reactivity over time. The chemical stability of a surface of the present invention may be determined using a form of the Arrhenius equation to predict the stability of the composition over time. The Arrhenius equation is used by those of skill in the art to predict the rates of chemical reactions and the stability of various thermolabile compounds as a function of temperature (U.S. Pat. No. 5,834,254). The Arrhenius equation assumes a first order reaction of reagent inactivation wherein an active reagent has a single rate of inactivation at a given temperature and a single mechanism of inactivation at all tested temperatures. The most preferred form of the equation, useful in the present invention is
ln(k₂/k₁)=(Eₐ/R)((T₂−T₁)/(T₂×T₁))
wherein k₂ equals the rate constant at the experimental temperature (° K), k₁ equals the rate constant for the reaction at a reference temperature, Eₐ equals the activation energy of the reaction, R equals the gas constant (1.987 cal/° K-mole), T₁ equals the reference temperature (e.g., 298.16° K (25° C.)), and T₂ equals the experimental temperature (expressed in ° K).

If Eₐ is assumed to be 15,000 cal/mol and the reference and experimental temperatures are known, then a ratio of the rate constants k₂/k₁ can be determined. In the simple case where both the reference and experimental temperatures are 25° C., the ratio of these constants is 1 since the constants are identical. If the experimental temperature is 35° C. and the reference temperature is 25° C., the predicted ratio will be 2.27. If the experimental temperature is 45° C. and the reference temperature is 25° C., the predicted ratio will be 4.91. Using the same equation, if the reference temperature is 5° C. and the experimental temperature is 45° C., the ratio is 30.33.

The rate constant ratios can be considered the “decomposition ratio” of the experimental storage time to the normal storage time, whether this time is expressed in hours, days, weeks, etc. Therefore, if the chemical reactivity of the surface of the invention decomposes to 90% of its original potency in 30 days at 45° C., the Arrhenius equation predicts that it would take 147.3 (30×4.91) days at 25° C. for the activity to be similarly reduced.

As used herein, “chemical stability”, as calculated using the Arrhenius equation refers to a loss of chemical reactivity of no less than 50%, 40%, 30%, 20%, or 10% of the original reactivity over a period of at least three months, four months, five months, and up to six months. It is preferred that a “chemically stable” surface of the present invention will not lose any chemical reactivity over at least three months, four months, five months, and up to six months.

As used herein, a “reagent containing a terminally unsaturated carbon” refers to a reagent selected from the group including, but not limited to acryloylchloride, acryloyliodide, acryloylbromide, 4-nitrophenyl-chloroformate, 4-nitrophenyl-bromoformate, 4-nitrophenyl-iodoformate, CH2=CRC(O)Cl, CHR═CHC(O)Cl, RR′C═CHC(O)Cl, CH2=CH(CH2)nC(O)Cl, wherein R or R′ comprises 1-15 carbons and n=0-10.

As used herein, “reacting” refers to a chemical reaction, wherein a “chemical reaction” refers to a chemical change that may occur in several ways, e.g., by combination, by replacement, by decomposition, or by some modification of these. “Chemical reactions” useful in the present invention may include, but may not be limited to oxidation, reduction, ionization, combustion, polymerization, hydrolysis, condensation, enolization, saponification, rearangement, and the like. Chemical reactions involve rupture of only the bonds which hold the molecules being reacted together, and should not be confused with nuclear reactions, in which the atomic nucleus is involved (Lewis et al., Hawley's Condensed Chemical Dictionary 12^{th} Ed. 1993 VanNostrand Reinhold NY, N.Y.)

As used herein, an “amine group containing compound” refers to a linear or branching compound composed of repeating or non-repeating units, comprising one or more amide linkage, or secondary or tertiary amines between repeating units or at the branching points, wherein the repeating or branching units comprised of carbon-carbon bonds, carbon-oxygen-carbon bonds or carbon-nitrogen-carbon bonds, terminated with primary amines at each or some of the end or the branch end. As used herein an “amine group containing compound” further refers to any organic molecule having the general formula NH₂(CH2)ₘY[(CH2)ₙY]ₓ(CH₂)ₘNH₂, NH₂(CH₂)ₙNH₂, wherein m, n equals 1-15, x equals 4-15, and Y is O, NH, CONH, N-HCO, NHCSNH, or NHCONH. Alternative “amine group containing compounds” include but are not limited to tetraethylenpentamine, 1,4-bis-(3-aminopropoxy)butane, 4-aminomethyl-1,8-octadiamine, 4,7,10-trioxa-1,13-tridecandiamine, N,N-dimethyl-1,6-hexadiamine, 2-(2-aminoethoxy)ethanol, jeffamine 130, 3-amino-1,2-propandiol, hesandiamine, cyclohexandiamine, pentaethylenehexaamine, polyethylenepolyamine, polypropylenimine hexadecaamine, polypropylenimine tetraamine dendrimer, polypropylenimine octaamine dendrimer, polypropylenimine hexadecaamine dendrimer, polypropylenimine dotriacontaamine dendrimer, and polypropylenimine tetrahexacontaamine dendrimer.

As used herein, a “reagent which activates amine groups” refers to any reagent which when contacted to an amino group having the formula NH₂, will react with the amino group such that the resulting reaction product can be covalently bonded to a substance comprising a hydroxyl group or amine group useful in the present invention. “Reagents which activate amino groups” include, but are not limited to phenylendiisothiocyanate, disuccinimidylcarbonate, phenylendiisocyanate, disuccinimidyloxalate, bis-2-succinimido-oxycarbonyloxyethyl sulfone (BSOCOES), sulfo-BSOCOES, bis-sulfosuccinimidyl-disuccinimidyl tartarate (DST), sulfo-DST, ethylene glycol bis-succinimidylsuccinate (EGS), dimethylsuberimidate, and other similar homo- or hetero-bifunctional cross linking reagents.

As used herein, a “substance comprising a hydroxyl group or amine group” refers to any molecule comprising a hydroxyl or amine group which is capable of participating in a chemical reaction with another molecule. “Substances comprising hydroxyl or amine groups” useful in the present invention include, but are not limited to nucleic acid, DNA, RNA, polynucleotides, oligonucleotides, and polypeptides.

As used herein, “polypeptide” refers to a series of amino acid residues linked through peptide bonds between the [symbol] carboxyl carbon of one amino acid residue and the [symbol] nitrogen of the next. A “polypeptide” as used herein further refers to proteins, or peptides.

As used herein, “polynucleotide” refers to a polymer of two or more nucleotide monomers or analogs thereof, and includes double- or single-stranded DNA, RNA or PNA (peptide nucleic acid).

As used herein, the term “oligonucleotide” refers to a polynucleotide that is between two and about 200 nucleotides in length. An oligonucleotide can be a synthetic (i.e., chemically synthesized) molecule, an enzymatically synthesized molecule or a naturally occurring molecule.

As used herein, “printing buffer” refers to any aqueous solution in which nucleic acid, protein, or polypeptides may be suspended for application onto the chemically reactive surface of the present invention. “Printing buffers” useful in the present invention include, but are not limited to 5% aqueous sodium bicarbonate (pH 8.4-8.5), 3×SSC, 50% DMSO, H₂O, 1% diisopropylethylamine, 1% N-methylmorphine, and 1% N-methylimidazole.

As used herein, “deactivating buffer” refers to any aqueous solution which may be used to treat the chemically reactive surface to block all still reactive functions on the surface, thus blocking any non-specific binding of nucleic acid, protein, or peptide in later stages of use of the surfaces of the present invention. “Deactivating buffers” useful in the present invention include, but are not limited to 5% aqueous sodium bicarbonate (pH 8.5-8.5), 50 mM 6-amino-hexanol-1, 1,3-diaaminopropane, 4-aminobutyric acid, 3-amino-1-propanol, 1-aminopropane, or any amino acid.

As used herein, “nucleic acid stripping buffer” refers to any aqueous solution which may be used to remove nucleic acid molecules hybridized to the “moieties comprising a hydroxyl group or amine group”, the nucleic acid or protein probes attached to the glass slide surface. According to the present invention, a chemically reactive surface may be reacted with a substance comprising a hydroxyl group or amine group. In one embodiment the moiety is nucleic acid. Subsequent to reacting the nucleic acid with the surface of the invention, the nucleic acid may be reacted with, for example, an oligonucleotide target. According to the invention the “nucleic acid stripping buffer” may be used to remove the oligonucleotide target from the nucleic acid bound reactive surface. A non-limiting example of a “nucleic acid stripping buffer” useful in the present invention is a aqueous solution comprising 2.5 mM Na₂HPO₄ and 0.1% (v/v) SDS.

### DESCRIPTION OF THE FIGURES

FIG. 1 shows a schematic diagram of the steps for constructing a chemically reactive surface of the present invention using a preformed dendrimer polyamine.

FIG. 2 shows a schematic diagram of the steps for constructing a chemically reactive surface of the present invention using a first amine group containing compound having the general formula: NH₂(CH2)ₘY[(CH2)ₙY]ₓ(CH₂)ₘNH₂, NH₂(CH₂)ₙNH₂, wherein m, n equals 1-15, x equals 4-15, and Y is O, or NH.

FIG. 3 shows the fluorescent images of (A) a poly-L-lysine coated slide and (B) a dendrimer polyamine coated slide.

FIG. 4 shows the digital fluorescent intensity of spots after background subtraction on poly-L-lysine coated slide (A) and dendrimer coated slide (B). The data is taken from the slides shown in FIGS. 3A and 3B when using sodium bicarbonate spotting buffer.

FIG. 5 shows a comparison between activated dendrimer polyamine coated slides stored for 98 days prior to DNA printing and a control slide which was printed the same day that it was activated.

FIG. 6 shows the digitized image analysis for DNA spot variation on dendrimer polyamine coated slides.

FIG. 7 shows three fluorescent images of dendrimer polyamine coated slides which have been hybridized with a cDNA probe, stripped, and re-hybridized for a total of three repetitions.

FIG. 8 shows the digitized image analysis of a dendrimer polyamine coated slide printed with cDNA which has been stored for 117 days prior to hybridization.

### DETAILED DESCRIPTION

The present invention provides a chemically reactive surface for binding a substance comprising a hydroxyl group or an amine group useful for binding a substance comprising a hydroxyl or amine group. In one embodiment, the substance comprises a —SH terminated oligonucleotide, DNA or protein. a preferred embodiment, the substance comprising a hydroxyl or amine group is DNA, RNA, protein, or peptide. In a further embodiment, the present invention provides a method of making a chemically reactive surface for binding moieties bearing hydroxyl or amine groups. The heterobifunctional crosslinker, coupling the nucleic acids or proteins, could be chosen so that sulfur containing compounds could be bound (i.e. —SH of cysteine residues).

Methods of Making the Chemically Reactive Surface

The present invention provides a method for making a chemically reactive surface comprising preferably the steps of: (a) contacting the surface with a silane containing moiety comprising a reactive functionality, wherein the moiety silanizes the surface, (b) contacting the silanized surface with a reagent containing a terminally unsaturated carbon capable of chemically reacting with the reactive functionality of the silanized surface, (c) reacting the silanized surface with a dendrimer polyamine (d) repeating steps (b) and (c) sequentially so as to generate a dendrimer polyamine of the desired generational size, and (e) reacting the dendrimer coated surface with a reagent which activates the amino groups so as to render the surface reactive with a substance comprising hydroxyl or amine groups.

Surfaces

Surfaces useful in the present invention include any two dimensional structure on a solid substrate which may be silanized and to which may subsequently be bound one or more dendrimer polyamines. In a preferred embodiment, the surface of the present invention is glass, and is preferably a glass microscope slide. Alternatively a surface, useful in the present invention may include, but may not be limited to poly-styrene, poly-carbonate or other plastic polymer slides, poly-styrene, poly-carbonate or other plastic polymer well plates, beads, membranes, glass wool, and any other solid support material which can support combinatorial chemistry reactions as known in the art.

In a preferred embodiment, prior to reacting a surface according to the invention with the silane containing moiety, the surface is examined under a standard dissecting microscope to ensure that there are no scratches, haze or imperfections on the surface. The surface is then subjected to a series of washing steps in aqueous solutions including, but not limited to ethanol, sodium hydroxide, hydrochloric acid, methanol, and water.

Silanization

Following the visual inspection and washing of the surface according to the invention, the surface is reacted with a silane containing moiety comprising a reactive functionality. The silane containing moiety may be a gaseous or liquid compound of silicon and hydrogen, wherein the moiety has the ability to bond to an inorganic substrate, such as the surfaces useful in the present invention. Silane containing moieties of the present invention have the general formula:
XR′Si(OR″)₃
where R′ is alkyl containing 0-10 carbon atoms, R″ is alkyl containing 1-10 carbon atoms, Si is a silicon, and X is referred to as the “reactive functionality”, and includes, but is not limited to NH₂, SH, OH, CN, halogens, methacrylate, quaternary amine salts, carboxylic acids and salts, phosphonates, succinic anhydride, 2-carbomethoxyaziridine, dihydroimidazole, thiocyanato, isocyanato, isopropeno, 2,3-epoxypropoxy, epoxy-alkyl, and the like. In a preferred embodiment, the reactive functionality (“X”) is NH₂.

In a preferred embodiment, the surface of the invention is reacted with the silane containing moiety at room temperature by sonication of the surface in an aqueous solution comprising the silane containing moiety and methanol, or other alcohol. The surface is then dried under a stream of compressed air, nitrogen, or other noble gas, and heated at between 100° and 120° C. for 15 minutes.

Acylation

After the surface has been dried and baked, it is reacted with a reagent containing a terminally unsaturated carbon which is capable of reacting with the reactive functionality of the silane containing moiety. The reagent containing a terminally unsaturated carbon may be selected from the group of reagents including, but not limited to acryloylchloride, acryloyliodide, acryloylbromide, 4-nitrophenyl-chloroformate, 4-nitrophenyl-bromoformate, 4-nitrophenyl-idodformate, CH2=CRC(O)Cl, CHR═CHC(O)Cl, or reagents having the general formulas:
RR′C═CHC(O)Cl, or CH2=CH(CH2)nC(O)Cl
wherein R or R′ comprises 1-15 carbons and n=0-10. In a preferred embodiment the reagent is acryloylchloride.

Reacting the surface with the reagent containing a terminally unsaturated carbon molecule generates a functionality on the surface to which an amine group containing reagent, such as a dendrimer polyamine, may be bound.

Reacting with Polyamine

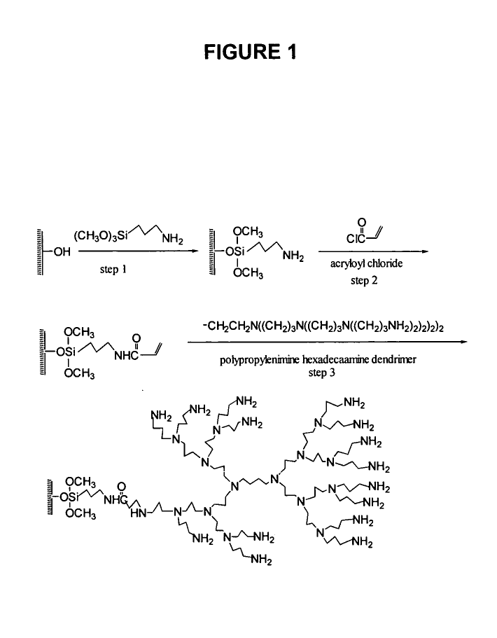

In a preferred embodiment the surface is subsequently reacted with a dendrimer polyamine comprising free terminal amine groups (see FIG. 1). A dendrimer polyamine is a macromolecular polymer with regular, dendritic branching with radial symmetry composed of an initiator core, interior layers (or generations) of repeating units radially attached to the core, and an exterior surface of terminal functional groups (See: D. A. Tomalia and H. D. Durst (1993) in E. Weber (ed.) Topics in Current Chemistry, Vol. 165: Supramolecular Chemistry I-Directed Synthesis and Molecular Recognition, Springer-Verlag, Berlin, pp.193-313). In a preferred embodiment, the terminal functional groups of dendrimer polyamines, useful in the present invention, are primary amine groups. In addition, dendrimer polyamines of the present invention useful in the present invention further comprise one or more amide linkages between successive dendrimer generations and secondary or tertiary amines at the branch points that do not comprise amide linkages, wherein the branch point comprises carbon-carbon bonds, carbon-oxygen-carbon bonds, or carbon-nitrogen-carbon bonds. A “dendrimer polyamine” useful in the present invention may include, but may not be limited to polypropylenimine hexadecaamine, polypropylenimine tetraamine dendrimer, polypropylenimine octaamine dendrimer, polypropylenimine hexadecaamine dendrimer, polypropylenimine dotriacontaamine dendrimer, and polypropylenimine tetrahexacontaamine dendrimer. In one embodiment, the dendrimer polyamine comprises [5]ₙ, [15]ₙ, [31]ₙ, or [63]ₙ terminal amino groups, wherein n=1, 2, 3, or 4. In one embodiment, dendrimer polyamines may be obtained from any commercial source known in the art (e.g., Sigma Aldrich, Milwaukee, Wis.). The surface is reacted with the dendrimer polyamine for between 48 and 72 hours in a solution containing the dendrimer polyamine and anhydrous, amine-free dimethylformamide (DMF) at room temperature.

In an alternate embodiment, the dendrimer polyamine may be fabricated stepwise on the surface. Following the reaction with a reagent containing a terminally unsaturated carbon, the surface is reacted with a first amine group containing compound having the general formula:
NH₂(CH₂)ₘY[(CH₂)ₙY]ₓ(CH₂)ₘNH₂,
wherein m,n equals 1-15, x equals 4-15, and Y is O, or NH. In a preferred embodiment, the first amine group containing compound is pentaethylenhexamine. Subsequent to reacting the surface with a first amine group containing compound, the surface is reacted a second time with the reagent comprising a terminally unsaturated carbon as described above. The surface is then reacted with a second amine group containing compound, selected from a group of compounds including, but not limited to tetraethylenpentamine, 1,4-bis-(3-aminopropoxy)butane, 4-aminomethyl-1,8-octadiamine, 4,7,10-trioxa-1,13-tridecandiamine, N,N-dimethyl-1,6-hexadiamine, 2-(2-aminoethoxy)ethanol, jeffamine 130, 3-amino-1,2-propandiol, hexandiamine, cyclohexandiamine, pentaethylenehexaamine, polyethylenepolyamine, and NH₂(CH₂CH₂NH)ₙCH₂CH₂NH₂, where n=2, 4, 5-12. The surface is reacted with either of the first or second amine group containing compound for between 48 and 72 hours in a solution containing the amine group containing compound and anhydrous, amine-free dimethylformamide (DMF) at room temperature.

Following reaction with either a dendrimer polyamine or amine group containing reagent as described above, the surface is washed in an aqueous solution including, but not limited to DMF, methanol and acetone. The surface is then dried under a stream of compressed air, nitrogen, or other compatible noble gas.

Surface Activation

The present invention provides a chemically reactive surface and a method of making such a surface comprising a dendrimer polyamine with free terminal amino groups for the attachment of a substance comprising free hydroxyl or amine groups. In order to covalently attach moieties comprising hydroxyl or amine groups to the modified surface, a chemical bond must be formed between the functional groups on the surface (i.e., NH₂) and the hydroxyl or amine groups on the moiety (i.e., DNA, RNA, protein, peptide). The present invention provides a bi-functional cross linking reagent to convert the dendrimeric terminal amine groups on the surface to their activated form. Accordingly, the surface is reacted for between 1-3 hours in an aqueous solution comprising an activating reagent, DMF, and anhydrous pyridine. Activating reagents useful in the present invention include, but are not limited to phenylendiisothiocyanate, disuccinimidylcarbonate, phenylendiisocyanate, disuccinimidyloxalate, bis-2-succinimido-oxycarbonyloxyethyl sulfone (BSOCOES), sulfo-BSOCOES, bis-sulfosuccinimidyl-disuccinimidyl tartarate (DST), sulfo-DST, ethylene glycol bis-succinimidylsuccinate (EGS), dimethylsuberimidate, and other similar homo- or hetero-bifunctional cross linking reagents. Subsequent to surface activation, the surface is washed with DMF and dichloroethane and then dried with compressed air. Once the surface is dry, it may be stored in its activated state at room temperature, and under anhydrous conditions for at least three months.

Surface Printing

The present invention provides achemically reactive surface to which may be bound one or more moieties comprising hydroxyl or amine groups. In one embodiment, the surface is reacted with DNA, RNA, protein, or peptide, wherein the DNA, RNA, protein or peptide is bound to the activated dendrimer polyamine of the invention, and wherein the surface may then be used in biochemical assays and the like (e.g., microarray screening assays). In a preferred embodiment, the DNA, RNA, protein, or peptide is unmodified, that is, no alterations have been made to the chemical structure following isolation, purification, or synthesis.

Nucleic Acid

Unmodified nucleic acid (DNA, RNA, cDNA, PCR product, or oligonucleotide) may be printed or attached to the activated dendrimer polyamine coated surface. Briefly, the nucleic acid is suspended in an aqueous solution comprising at least sodium bicarbonate at a concentration of between 0.5 and 0.0125 [symbol] g/ [symbol] nucleic acid. Techniques and methods for the printing and addressing of microarray slides is well known in the art (see, for example, U.S. Pat. Nos. 5,412,087; 5,837,832). The nucleic acid solution may be applied to the surface manually using hand-held glass capillaries. Alternatively, the nucleic acid may be applied to the activated surface using a robotic apparatus equipped with fine pin-tools or a piezoelectric-driven dispensing system (BioGrid, BioRobotics, UK; Nanoplotter, GeSiM, Germany). Regardless of the technique used, the application of nucleic acid onto the activated surface is performed at room temperature under humidified conditions (50-90% humidity). Following printing, the surface is incubated at room temperature and humidified conditions for 36 to 48 hours. The surface must subsequently be deactivated to block all remaining reactive functions on the surface, thus blocking any non-specific binding of nucleic acid, protein, or peptide in later stages of use of the surfaces of the present invention. The surface is incubated in a deactivating buffer for 1-3 hours at room temperature. Deactivating buffers useful in the present invention include, but are not limited to 5% aqueous sodium bicarbonate (pH 8.5-8.5), 50 mM 6-amino-hexanol-1, 1,3-diaminopropane, 4-aminobutyric acid, 3-amino-1-propanol, or 1-aminopropane. The surface is then dried with compressed air, nitrogen or any other compatible noble gas.

Protein and Peptide

In one embodiment of the present invention provides a chemically reactive surface to which may be bound a substance comprising hydroxyl amine or —SH groups such as proteins or peptides. Unmodified protein or peptide may be printed or attached to the activated dendrimer polyamine coated surface. Briefly, the protein or peptide is suspended in an aqueous solution comprising at least either sodium bicarbonate (pH 8-10) or sodium borate (pH 8-10) at a concentration of between 0.5 and 0.0001 [symbol] g/ [symbol] . The protein or peptide solution may be spotted, activated, and deactivated using the same or analogous methods to those described above for the printing of nucleic acid.

Surface Stability

Activated chemically reactive surfaces produced by the methods of the present invention or surfaces printed with nucleic acid, peptide or protein according to the invention, are chemically stable at room temperature, under anhydrous conditions for at least three months. In a preferred embodiment of the present invention, surfaces constructed according to the present invention are chemically stable at room temperature for at least ten months. The chemical stability of a surface of the present invention may be determined using a form of the Arrhenius equation to predict the stability of the composition over time. The Arrhenius equation is used by those of skill in the art to predict the rates of chemical reactions and the stability of various thermolabile compounds as a function of temperature (U.S. Pat. No. 5,834,254). The Arrhenius equation assumes a first order reaction of reagent inactivation wherein an active reagent has a single rate of inactivation at a given temperature and a single mechanism of inactivation at all tested temperatures. The most preferred form of the equation, useful in the present invention is
ln(k₂/k₁)=(Eₐ/R)((T₂−T₁)/(T₂×T₁))
wherein k₂ equals the rate constant at the experimental temperature (° K), k₁ equals the rate constant for the reaction at a reference temperature, Eₐ equals the activation energy of the reaction, R equals the gas constant (1.987 cal/° K-mole), T₁ equals the reference temperature (e.g., 298.16° K (25° C.)), and T₂ equals the experimental temperature (expressed in ° K.).

If Eₐ is assumed to be 15,000 cal/mol and the reference and experimental temperatures are known, then a ratio of the rate constants k₂/k₁ can be determined. In the simple case where both the reference and experimental temperatures are 25° C., the ratio of these constants is 1 since the constants are identical. If the experimental temperature is 35° C. and the reference temperature is 25° C., the predicted ratio will be 2.27. If the experimental temperature is 45° C. and the reference temperature is 25° C., the predicted ratio will be 4.91. Using the same equation, if the reference temperature is 5° C. and the experimental temperature is 45° C., the ratio is 30.33.

The rate constant ratios can be considered the “decomposition ratio” of the experimental storage time to the normal storage time, whether this time is expressed in hours, days, weeks, etc. Therefore, if the chemical reactivity of the surface of the invention decomposes to 90% of its original potency in 30 days at 45° C., the Arrhenius equation predicts that it would take 147.3 (30×4.91) days at 25° C. for the activity to be similarly reduced.

Kits

The present invention provides a kit comprising an anhydrous, activated chemically reactive surface, made according to the present invention, to which may be bound one or more moieties comprising hydroxyl or amine groups and packaging materials therefore. In a preferred embodiment, the kit comprises an anhydrous, activated reactive surface comprising a dendrimer polyamine having [5]ₙ, [15]ₙ, [31]ₙ, or [63]ₙ terminal amino groups, wherein n=1, 2, 3, or 4. The kit may also comprise nucleic acid or polypeptide printing buffer for reacting nucleic acid, peptide or protein with the activated surface as described above. The kit may also include a deactivating buffer which may include, for example, 5% aqueous sodium bicarbonate (pH 8.5-8.5), 50 mM 6-amino-hexanol-1,1,3-diaminopropane, 4-aminobutyric acid, 3-amino-1-propanol, 1-aminopropane, or any amino acids.

In an alternate embodiment, the invention provides a kit comprising a chemically reactive surface on which is printed DNA, RNA, protein, or peptide, made according to the present invention. The kit may also comprise a nucleic acid hybridization buffer. The composition of buffers for the hybridization of nucleic acids is well known in the art (see, for example, Ausubel et al., Short Protocols in Molecular Biology 3^{rd} Ed. John Wiley & Sons, Inc. 1995). For example, the nucleic acid hybridization buffer may include, but may not be limited to 0.1% SDS, 3×SSC, yeast tRNA, and salmon sperm DNA. The kit may also comprise a nucleic acid stripping buffer which may be used to remove nucleic acid which has been hybridized to the nucleic acid bound to the dendrimer polyamine of the surface. For example, a nucleic acid stripping buffer may include 2.5 mM Na₂HPO₄ and 0.1% SDS.

### EXAMPLES

### Example 1

### Production of Chemically Reactive Surfaces

Surface Modification with Preformed Dendrimer Polyamine

In a dust free area, 120 glass slides were placed into 6 polypropylene slide racks. The slides were examined to ensure there were no scratches, haze or other imperfections of the glass slides using the dissecting microscope. The slide racks were put into a 1.5 L polypropylene container and washed overnight with 100% ethanol with agitation. After rinsing with water, the slides were immersed in 10% aqueous sodium hydroxide solution overnight, followed by rinsing with water, 1% hydrochloric acid, water and then 100% methanol. After a 15 minute sonication in 1.5 L 95% methanol containing 3% aminopropyl trimethoxysilane, the slides were washed in methanol, water, and then dried under a stream of compressed air and heated at 110° C. for 15 minutes. The amine-silanized glass slides were incubated in a polypropylene container at room temperature for 2 hours in 1.5 L anhydrous dichloroethane containing 0.27% acryloylchloride and 0.57% diisopropylethyl-amine (DIEA). Subsequently, the slides were thoroughly washed with dichloroethane (1 L, repeat 3 times) then dried. The acylated slides were incubated for 48 hours in 1.5 L of anhydrous, amine-free dimethylformamide (DMF) containing 0.74% of polypropylenimine hexadecaamine dendrimer (FIG. 1) . Afterwards, the slides were extensively washed for 5 minutes with DMF, methanol and acetone (1 L each) then dried under a stream of compressed air.

Surface Modification with Amine Group Containing Compound

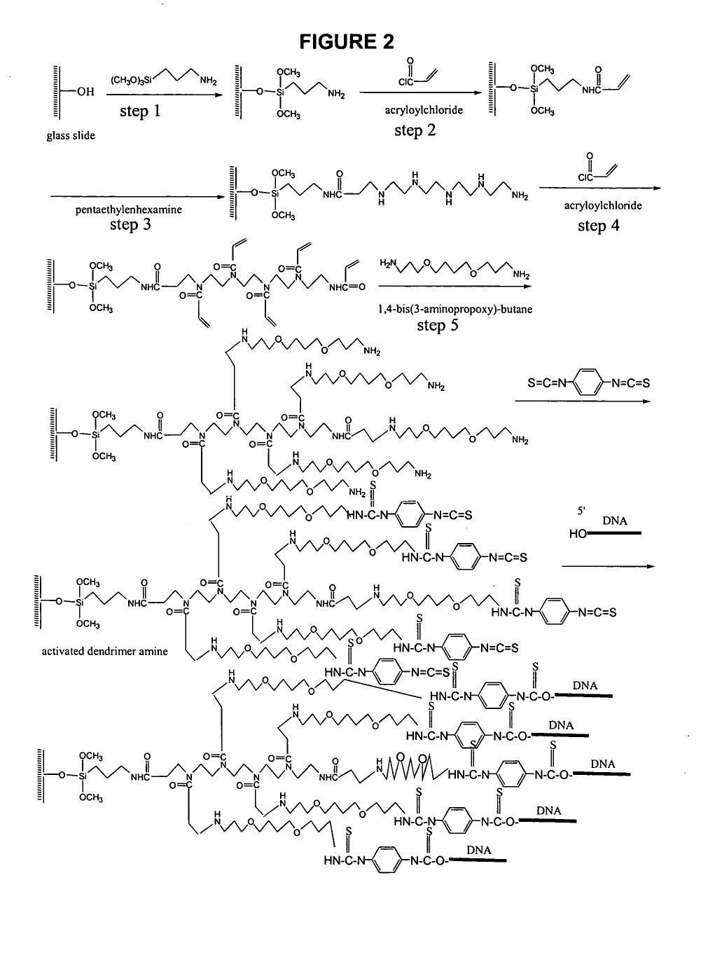

In a dust free area, 120 glass slides were placed into 6 polypropylene slide racks. The slides were examined to ensure there were no scratches, haze or other imperfections of the glass slides using the dissecting microscope. The slide racks were put into a 1.5 L polypropylene container and washed overnight with 100% ethanol with agitation. After rinsing with water, the slides were immersed in 10% aqueous sodium hydroxide solution overnight, followed by rinsing with water, 1% hydrochloric acid, water and then 100% methanol. After a 15 minute sonication in 1.5 L 95% methanol containing 3% aminopropyl trimethoxysilane, the slides were washed in methanol, water, and then dried under a stream of compressed air and heated at 110° C. for 15 minutes. The amine-silanized glass slides were incubated in a polypropylene container at room temperature for 2 hours in 1.5 L anhydrous dichloroethane containing 0.27% acryloylchloride, and 0.57% (DIEA). Subsequently, the slides werw thoroughly washed with dichloroethane (1 L, repeat 3 times) then dried. The acylated slides were incubated for 48 hours in 1.5 L of anhydrous, amine-free dimethylformamide (DMF) containing 0.74% of pentaethylenhexamine (FIG. 2). Afterwards, the slides were extensively washed with DMF, methanol and acetone before being dried under a stream of compressed air. The aminated glass slides were incubated for 3 hours in 1.5 L anhydrous dichloroethane containing 0.27% acryloylchloride, and 0.57% (DIEA). After washing with dichloroethane, the slides were incubated for 72 hours in 1.5 L of anhydrous, amine-free DMF containing 0.74% of 1,4-bis-(3-aminopropoxy)butane, washed for 5 minutes with DMF, methanol and acetone (1 L each) then dried under a stream of compressed air.

Activation of the Surface

The amine functionalized glass slides generated according to either of the above two methods were reacted for 2 hours in 1.5 L of 10% anhydrous pyridine in DMF containing 0.5% (w/v) of 1,4-phenylenediisothiocyanate. The reaction was carried out in a polypropylene container. Subsequently, the slides were washed for 5 minutes with DMF and dichloroethane (1 L each) and then dried with compressed air.

### Example 2

### DNA Printing and Slide Deactivating

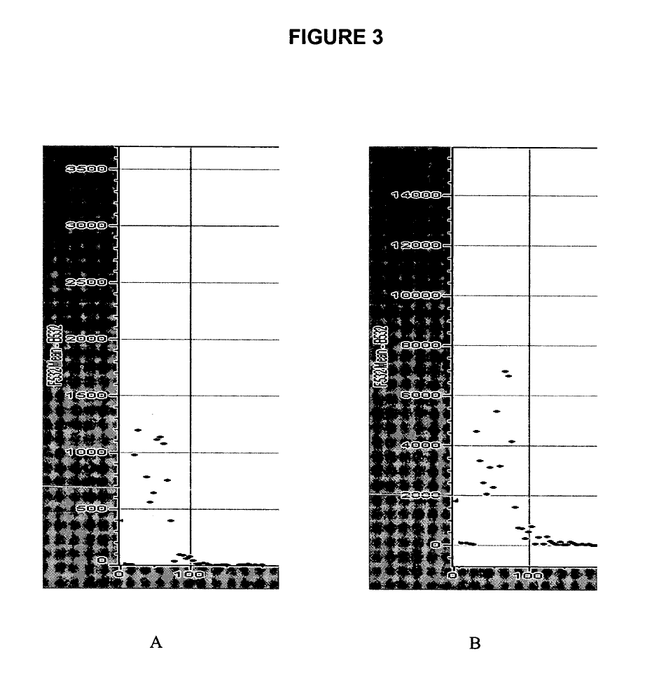

Unmodified probe DNA (specifically, [symbol] actin, X56062, X14212, U91966, ssDNA, Cot 1 DNA, and a 73-mer oligonucleotide; see FIG. 3) were suspended in 5% aqueous sodium bicarbonate solution (pH 8.4-8.5) at a concentration between 0.5-0.0125 μg/μl. Preactivated dendrimer amine slides are loaded on an OmniGrid arrayer (GeneMachines San Carlos, Calif.) and the arraying chamber was brought to 25° C. with a humidity of 70-80%. After array printing, the slides were incubated at 37° C. for 36 hours at a humidity of 70-80%. The slides were rinsed with water and 100% methanol, then deactivated in 1.5 L DMF solution containing 40 mL of diisopropylethylamine and 8.76 g of 6-amino-hexanol-1 for 2 hours at room temperature. The slides were rinsed for 5 minutes with DMF, acetone and water (1 L each). The deactivating could also be carried out by the reaction of printed slides with 5% aqueous sodium bicarbonate (pH 8.4-8.5) or 5% of 4-aminobutyric acid in 5% aqueous sodium bicarbonate (pH 8.3) for 2 hours at room temperature. The DNA array slides were dried by air stream and packaged in sealed bags.

### Example 3

### Hybridization to DNA Arrays

For hybridization with target oligonucleotides, fluorescently-labeled oligomer target (specifically, Cy3-labeled cDNA made from 33 μg of total HeLa RNA, or Cy5-labeled 73-mer (100 ng)) was put into 10 μl of solution containing 0.1% SDS, 0.8 μg/μl of polydA(40-60), 3×SSC, 0.4 μg/μl of yeast tRNA, 1 μg/μl of human Cot-1 DNA, 1 μg/μl of salmon sperm DNA. To generate the cDNA targets, RT/PCR products were labeled with 5′-Cy3 or Cy5 primers, or Cy dye labeled dUTP using standard protocols (Microarray Labeling Kit, Stratagene, La Jolla, Calif.). Starting with 10 to 33.3 μg of total RNA, the labeled cDNA target was purified by BioRad Spin-6 column (BioRad) or Microcon P-6 column, mixed in 10 μl of a solution containing 0.1% SDS, 0.8 μg/μl of polydA(40-60), 3×SSC, 0.4 μg/μl of yeast tRNA, 1 μg/μl of human Cot-1 DNA, 1 μg/μl of salmon sperm DNA. Hybridization was carried out in a humid chamber or under a cover slip (22×22 mm). The hybridization temperature was determined by the length of the target: 30-73 mer at 42° C., and PCR products at 60° C. After 17 hours, the slides were washed for 5 minutes with 500 mL of 0.5×SSC, 0.1% SDS at room temperature then 0.06×SSC for 5 minutes at room temperature. After drying under a stream of compressed air, the slides were scanned using an Axon Scanner (Axon Instruments, Inc.).

### Example 4

### Dendrimer Slide Surface Characteristics

Reactive amino groups were covalently attached to the glass slide surface, and used to create a dendritic polyamine structure through several steps as described above. The total number of free amines on the slide surface is 5 to 15 fold as much as on the traditional poly-lysine slide, by using the methods shown in FIGS. 1 and 2. The modified slides with free primary amines were then treated with a bi-functional cross linking reagent, such as phenylene diisothiocyanate, forming a pre-activated three dimensional platform suitable for attachment of DNA through 5′-hydroxyl groups. The pre-activated platform was stable at room temperature for more than 3 months. The loading capacity of probe DNA was increased due to the higher amine density of the dendrimer structure, and signals observed were 5 times or more higher than polylysine (FIG. 3). Since the dendrimer polyamine is covalently bonded to the glass surface, and the probe DNA is covalently bonded to the dendrimer as well, the probe DNA on surfaces according to the invention are more stable than on other printing surfaces that rely on non-covalent coupling procedures.

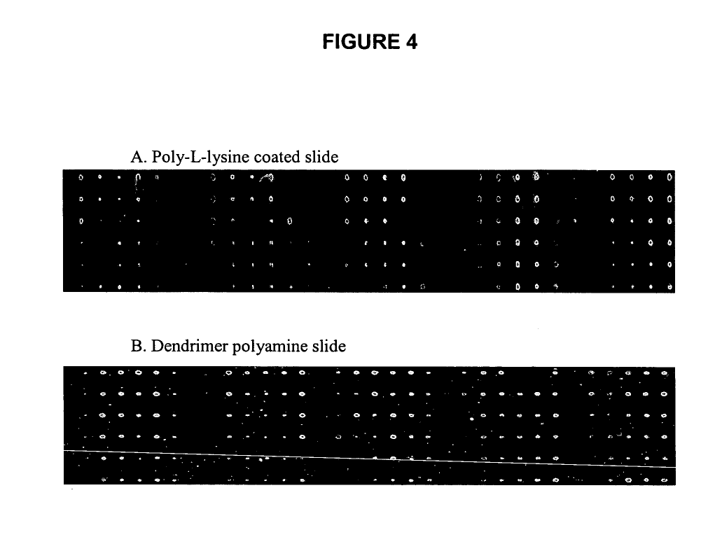

FIG. 4 shows the fluorescent images of (A) poly-L-lysine slide and (B) dendrimer polyamine slide. Each slide was spotted with (from right column to left) β-actin, X56062, X14212, U91966, ssDNA, Cot 1 DNA, oligo 73-mer and then repeated. Different buffers were used for spotting: column 1 (from the right) to 7, 3×SSC; column 8-14, 1% diisopropylethylamine in water; column 15 to 21, 10% sodium bicarbonate; column 22 to 28, water; column 29 to 35, 3% DMSO in water. DNA spotting concentration ranges from 0.5 μg/μl (top row), 0.25, 0.125, 0.0625, 0.0313 to 0.0156 (bottom row) μg/μl. Each slide was hybridized with Cy3-labeled cDNA made from 33 μg of total HeLa RNA and Cy5-labeled 73 mer (100 ng) target individually, then the image was overlapped. Clear and strong signals with very low background are obtained from the dendrimer slides of the invention (FIG. 3B). There is no signal streaking between spots and no coating damage exists as frequently seen in polylysine slides (FIG. 3A). As shown in FIG. 3A, probe loading concentration higher than 0.125 μg/μl (third row down from the top) leads to signal streaking on polylysine slide, while on the dendrimer slide the signal is clear and strong within the whole concentration range.

### Example 5

### Storage and Shelf-Life Testing

Pre-activated dendrimer amine slides or DNA printed slides were put into a slide rack, sealed in a plastic/aluminum bag with desiccant and stored at room temperature. An accelerated stability study was performed utilizing the Arrhenius equation to predict the room temperature stability of the slides (U.S. Pat. No. 5,834,254). A few packages of slides were kept in ovens at 30° C. or 45° C. After several weeks at 30° C. or 45° C., the slides were removed from the oven, printed with cDNA target and the slides were deactivated as described above. The slides were then hybridized with Cy5-labeled cDNA. The stability of pre-activated dendrimer polyamine slides was evaluated by comparing the spot size, shape, image and the background level compared with the positive control (i.e., slides which were activated and immediately printed probe DNA).

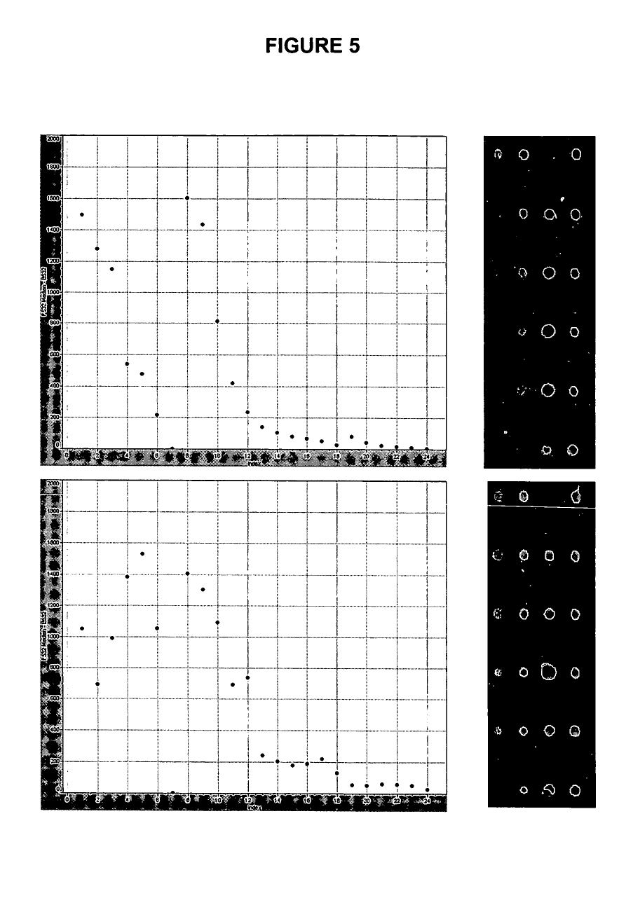

The results of the slide stability testing is shown in FIG. 5. FIG. 5 shows the intensity and image of dendrimer polyamine slides printed with (from right to left column): β-actin, X56062, X14212, U91966 with concentrations from 0.5 (top), 0.25, 0.125, 0.063, 0.03 to 0.125 μg/μl. The top slide was printed the same day that the amine groups were activated. The bottom slide was pre-activated and stored for 98 days at room temperature, then printed with a cDNA probe. The slides were hybridized with Cy3 labeled cDNA made from 33 μg of total HeLa RNA. No changes are observed in spot intensity, size, shape and background level (FIG. 5B). This experiments demonstrates the stability of the preactivated dendrimer polyamine slide for at least three months at room temperature.

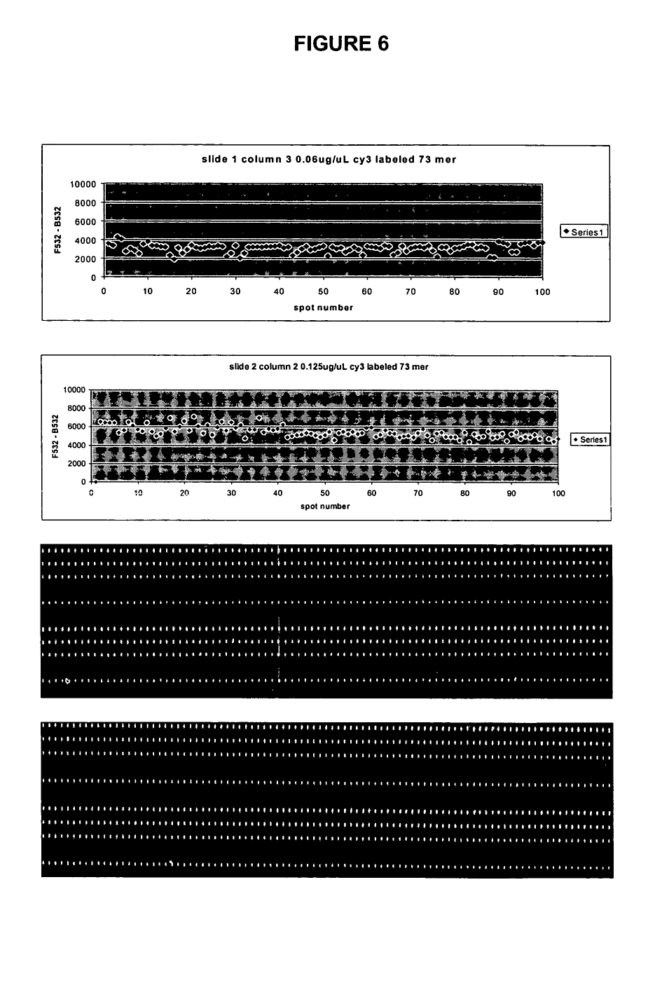

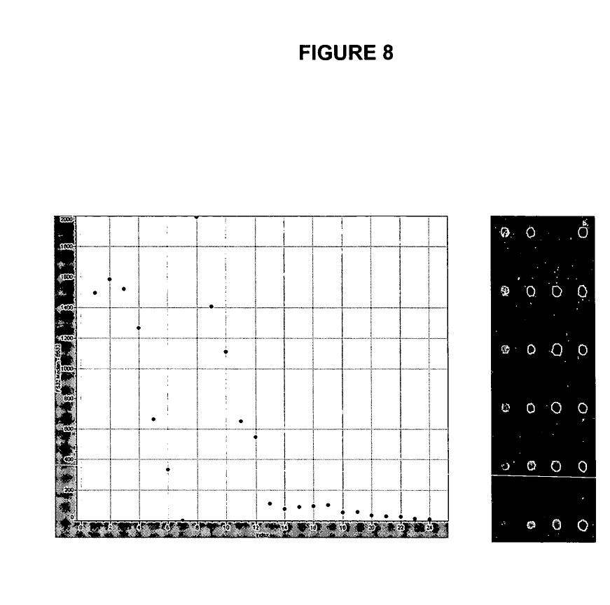

In addition to the pre-activated dendrimer polyamine slides, we also studied the shelf-life of pre-printed cDNA slides. Probe cDNA was printed on dendrimer slides, slides were deactivated then sealed in bags with desiccant. After 24 days incubation at 45° C., the slides were cooled to room temperature, hybridized with Cy3 labeled cDNA made from 33 μg of total HeLa RNA. No difference in spot size, shape, image intensity and background level (FIG. 8) was found in these slides compared with positive control in FIG. 6, indicating that the probe DNA immobilized on the preactivated dendrimer amine is stable at room temperature for at least 117 days.

### Example 6

### Printing Reproducibility of Pre-Activated Dendrimer Polyamine Slides

One of the critical factors for creating satisfactory slides is the creation of uniform, consistent and reproducible spots. Spot variation results from mechanical differences between spotting pins, slight variations in slide surface properties, and changes in the pin during the printing process due to clogging. This variation was measured by printing Cy3 labeled oligonucleotide (30 mer) on dendrimer polyamine slides produced according to the present invention. A series of Cy3-30 mer spots with a concentration ranging from 0.25 to 0.03 μg/μl were printed onto 5 slides. Digitized spots indicated that spot size, shape and intensity are very similar to each other within a single slide and with different slides with the same concentration pin The coefficient of variation (c.v.) for spots across the 5 slides ranges from 14.4% to 15.2%, indicating a very satisfactory uniformity (FIG. 5). The reported c.v. for most commercial slides is around 16% (Schena Ed., Microarray Biochip Technology 2000, Eaton Publishing, Mass.).

### Example 7

### Reusability

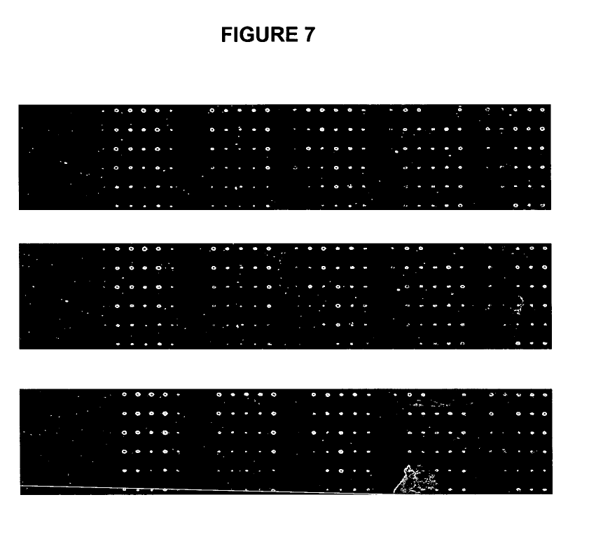

Dendrimer polyamine slides were constructed according to the methods of the present invention. FIG. 7 shows three fluorescent images of a dendrimer polyamine slide. The slide was spotted with (from right column to left) β-actin, X56062, X14212, U91966, ssDNA, Cot 1 DNA, oligo 73-mer and then repeated. Different buffers were used for spotting: column 1 (from the right) to 7, 3×SSC; column 8-14, 1% diisopropylethylamine in water; column 15 to 21, 10% sodium bicarbonate; column 22 to 28, water; column 29 to 35, 3% DMSO in water. DNA spotting concentration ranges from 0.5 μg/μl (top row), 0.25, 0.125, 0.0625, 0.0313 to 0.0156 μg/μl (bottom row). Each slide was hybridized with Cy3-labeled cDNA made from 33 μg of total HeLa RNA and Cy5-labeled 73 mer (100 ng) target individually, then the image was overlapped. The slide was reused three times by stripping off the target and rehybridizing with a Cy3-cDNA and Cy5-73 mer. Since the dendrimer polyamine is covalently bonded to the slide surface and the probe DNA is covalently attached to the dendrimer as well, DNA arrays made according to the methods of the present invention can be reused at least three times without loss of signal intensity or clarity (FIG. 7). The dendrimer treated DNA arrays of the invention may be used for at least three “biochemical reactions”, wherein biochemical reactions, useful in the present invention include nucleic acid hybridization as described above, antibody/antigen reactions, or any other biochemical or chemical reaction performed on the moiety comprising a hydroxyl or amine group covalently bound to the dendrimer microarray of the invention.

### OTHER EMBODIMENTS

Other embodiments will be evident to those of skill in the art. It should be understood that the foregoing detailed description is provided for clarity only and is merely exemplary. The spirit and scope of the present invention are not limited to the above examples, but are encompassed by the following claims.

1. A chemically reactive surface which is reactive with a substance comprising a hydroxyl group or amine group, wherein said chemically reactive surface comprises a solid surface and an activated dendrimer polyamine, wherein said solid surface is covalently bound through a silane containing moiety to said activated dendrimer polyamine, wherein said dendrimer polyamine comprises branch points and terminal residues, a said branch point of said dendrimer comprising either a secondary or a tertiary amine, a said terminal residue comprising a moiety selected from the group consisting of primary amine, hydroxyl, carboxyl, and thiol, and wherein in the presence of a substance comprising a hydroxyl group or amine group, the terminal groups of said activated dendrimer polyamine covalently bind said substance through said hydroxyl group or amine group.

2. The surface of claim 1, wherein said solid surface is glass.

3. The glass surface of claim 2, wherein said glass surface is a glass slide.

4. The chemically reactive surface of claim 1, wherein said silane containing moiety has the formula
XR′Si(OR″)₃
wherein R′ is alkyl containing 0-10 carbons, R″ is alkyl containing 1-10 carbons, X is selected from the group consisting of NH₂, SH, OH, CN, halogen, methacrylate, quaternary amine salt, carboxylic acid and carboxylic acid salt, phosphonate, succinic anhydride, 2-carbomethoxyaziridine, dihydroimidazole, thiocyanato, isocyanato, isopropeno, 2,3-epoxypropoxy, and epoxy-alkyl.

5. The chemically reactive surface of claim 1, wherein said dendrimer polyamine comprises ⁿ terminal primary amine groups, wherein n=1, 2, 3, or 4.

6. The chemically reactive surface of claim 1, wherein said chemically reactive surface further comprises said substance comprising a hydroxyl group or amine group, wherein said substance is selected from the group consisting of DNA, RNA, and polypeptides.

7. The chemically reactive surface of claim 1, wherein said chemically reactive surface further comprises said substance comprising a hydroxyl or amine group, wherein said substance is covalently bonded to said activated dendrimer polyamine.

8. The chemically reactive surface of claim 1, wherein said chemically reactive surface is chemically stable at room temperature.

9. The chemically reactive surface of claim 8, wherein said chemically reactive surface is chemically stable at room temperature for at least three months.

10. The chemically reative surface of claim 1, wherein the terminal amine groups of said activated dendrimer polyamine are converted to their activated form with a bifunctional cross linking reagent to render said activated dendrimer polyamine reactive with a substance comprising hydroxyl or amine groups through said hydroxyl or amine groups of said substance.

11. The chemically reative surface of claim 1, wherein the bifunctional crosslinking reagent is selected from the group consisting of phenylendiisothiocyanate, disuccinimidylcarbonate, phenylendiisocyanate, disuccinimidyloxalate, bis-2-succinimido-oxycarbonyloxyethyl sulfone (BSOCOES), sulfo-BSOCOES, bis-sulfosuccinimidyl-disuccinimidyl tartarate (DST), sulfo-DST, ethylene glycol bis-succinimidylsuccinate (EGS) ad dimethylsuberimidate.
